(19) United States
(12) Patent Application Publication
(10) Pub. No.: US 2013/0109920 A1
(43) Pub. Date: May 2, 2013

(54) ARRANGEMENT AND METHOD FOR NAVIGATING AN ENDOSCOPIC CAPSULE

(71) Applicants: Mario Bechtold, Hemhofen (DE); Christoph Gabriel, Erlangen (DE); Aleksandar Juloski, Nuernberg (DE)

(72) Inventors: Mario Bechtold, Hemhofen (DE); Christoph Gabriel, Erlangen (DE); Aleksandar Juloski, Nuernberg (DE)

(21) Appl. No.: 13/458,937

(22) Filed: Apr. 27, 2012

(30) Foreign Application Priority Data
Apr. 28, 2011 (DE) 10 2011 017 719.1

(51) Int. Cl. A61B 1/00

(52) U.S. Cl. CPC A61B 1/00131
USPC 600/118

(57) ABSTRACT

An arrangement for navigating an endoscopic capsule includes an external first magnetic field with a first magnetic flux density for moving the endoscopic capsule. The arrangement also includes at least one sensor coil pair outside of the endoscopic capsule for determining a position and/or orientation of the endoscopic capsule. The at least one sensor coil par includes a first sensor coil and a second sensor coil. The first sensor coil and the second sensor coil are electrically connected to one another and arranged at locations with the same first magnetic flux density.

This application claims the benefit of DE 10 2011 017 719.1, filed on Apr. 28, 2011.

### BACKGROUND

The present embodiments relate to an arrangement and a method for navigating an endoscopic capsule.

Conventional endoscopy is a widespread process within medicine, both for the examination and diagnosis and also treatment and therapy of a patient. With conventional endoscopy, an endoscope and/or catheter is introduced into a hollow organ of the patient (e.g., the stomach or intestine) via an orifice in the patient (e.g., the mouth or the rectum).

Conventional endoscopes are disadvantageous (e.g., exhibit a limited range from the orifice of the patient to the interior of the body and/or a limited flexibility in terms of tracing curves or loops in hollow organs). For example, it may not be possible to completely reach the small intestine of a patient with a length of, for example, 7 to 8 meters using a conventional endoscope of this type.

Endoscopy systems with magnetically controlled endoscopic capsules therefore exist for improved examination of the entire length of the stomach/intestinal tract. A capsule may be approximately 30 mm long and may have a diameter of approximately 10 mm. Images are taken by way of integrated miniature cameras and a transmit/receive electronics unit and are transmitted in a contact-free fashion to a display and evaluation unit. A magnetically controlled endoscopic capsule is described, for example, in the patent application DE 101 42 253 C1.

The magnet guidance is achieved by magnetic forces and torque due to magnetic gradient fields that act on a permanent magnet in the capsule. The magnetic gradient field is generated by an external driving magnet. The external driving magnet may be an electromagnet, such as is described, for example, in the application WO 2006/092421. The driving magnet may alternatively contain one or several mechanically moveable permanent magnets. The magnetization direction of the permanent magnets of the capsule may lie perpendicular to the longitudinal axis of the capsule.

The magnetic forces and torque on the capsule are proportional to the size of the permanent magnet in the capsule and to the electric current in the coils of the driving magnets. Whereas the size of the permanent magnet is restricted by the capsule size, the coil currents are restricted by the power supply and the heat development.

The position and orientation of an endoscopic capsule in the human body must be known in order to be able to adjust the driving magnets. The position determination method is based on magnetic induction. A marker coil arranged in the endoscopic capsule generates an electromagnetic alternating field that induces a voltage into sensor coils outside of the human body. On account of the different voltages in the different sensor coils, the position and orientation of the endoscopic capsule may be calculated. A measuring arrangement of this type is described in the application EP 1 967 137 A1.

In order to generate the electromagnetic alternating field, the marker coil is supplied with alternating current. The alternating current is either generated by an external alternating current source and with the aid of the magnetic induction or with a battery in the capsule and a circuit connected thereto. On account of spatial restrictions, the sensor coils may lie within the driving magnet coils. A strong magnetic coupling results between the driving magnet coils and the sensor coils. The driving magnet coils, into which large currents flow in order to navigate the capsule, induce voltage into the sensor coils. As a result, either an evaluation electronics unit downstream of the sensor coils is driven to saturation, or the input dynamic range of the evaluation electronics unit is extended such that the level of any induced voltages that are attributable to the marker coil only use the dynamic range to a minimal degree.

The capsule may not have a self-sufficient power supply, but is instead supplied with electrical energy by an external energy coil. The energy coil is operated with the same frequency as a resonance circuit of the marker coil. The energy coil generates a magnetic field that also generates a voltage in the sensor coils. As a result, current flows. The driving magnet coils may drive the evaluation electronics unit of the sensor coils to saturation. Since the energy coil also generates a strong alternating magnetic field, the problem of the necessary great dynamic range of the evaluation electronics unit exists solely on account of the energy supply coil.

US 2010/0134096 A1 discloses an arrangement for determining the position of a capsule using a coil in a magnetic field. The magnetic field generated by the coil of the capsule may be detected with the aid of an array of sensor coils.

DE 10 2005 053 759 A1 discloses a method and a facility for wirelessly transmitting power from a magnetic coil system including several exciter coils outside of a patient to a working capsule in the patient including at least one induction coil. A location facility determines a position and orientation of the working capsule relative to the magnetic coil system, and, on the basis of the position and orientation, the magnetic coil system generates a first magnetic field at the site of the working capsule to exert force on the working capsule. On the basis of the position and/or orientation, the magnetic coil system generates a second magnetic field at the site of the working capsule to transfer energy to the working capsule.

### SUMMARY AND DESCRIPTION

The present embodiments may obviate one or more of the drawbacks or limitations in the related art. For example, an arrangement and a method for navigating an endoscopic capsule that reduce the influence of external magnetic fields on sensor coils are provided.

With a counterpolar electrical connection of a coil pair, voltages induced into the coils by an external magnetic field are cancelled out if the magnetic field strengths at the site of the coils are of equal magnitude. As a result, the effect of an external magnetic field may be compensated.

An arrangement for navigating an endoscopic capsule with an external first magnetic field having a first magnetic flux density for moving the endoscopic capsule is provided. The arrangement includes at least one sensor coil pair outside of the endoscopic capsule for determining a position and/or orientation of the endoscopic capsule. The coil pair includes a first sensor coil and a second sensor coil. The first sensor coil and the second sensor coil are electrically connected to one another and are arranged at locations with the same first magnetic flux density. The present embodiments are advantageous in that voltages induced in the coil pair by the interfering magnetic fields are compensated. Only the non-symmetrical voltages of a wanted signal are available at an input to a sensor electronics unit. The wanted signal of several sensor coil pairs is a measure of the position and orientation of an endoscopic capsule.

An arrangement for navigating an endoscopic capsule with an external second magnetic field having a second magnetic flux density for supplying power to a marker coil of the endoscopic capsule is also provided. The arrangement includes at least one sensor coil pair outside of the endoscopic capsule for determining a position and/or orientation of the endoscopic capsule. The at least one sensor coil pair includes a first sensor coil and a second sensor coil. The first sensor coil and the second sensor coil are electrically connected to one another and are arranged at locations with the same second magnetic flux density.

In one embodiment, the first sensor coil and the second sensor coil are connected to one another such that voltages induced by the first magnetic field are compensated. In another embodiment, the first sensor coil and the second sensor coil may be connected to one another such that the voltages induced by the second magnetic field are compensated. The dynamic range of a sensor electronics unit for evaluating inducted sensor voltages may be selected appropriately since, the dynamic range is not determined by interfering magnetic

The arrangement may include a driving coil that generates the first magnetic field. The first sensor coil and the second sensor coil are arranged symmetrically relative to a coil axis of the driving coil.

In one embodiment, the arrangement may include an energy coil that generates the second magnetic field. The first sensor coil and the second sensor coil are arranged symmetrically relative to the coil axis of the energy coil.

A method for navigating an endoscopic capsule through an external first magnetic field having a first magnetic flux density to move the endoscopic capsule is also provided. The method includes a determination of a position and/or orientation of the endoscopic capsule. At least one sensor coil pair is arranged outside of the endoscopic capsule. The at least one sensor coil pair includes a first sensor coil and a second sensor coil. The first sensor coil and the second sensor coil are electrically connected to one another and are arranged at locations with the same first magnetic flux density.

A method for navigating an endoscopic capsule through an external second magnetic field having a second magnetic flux density for supplying power to a marker coil of the endoscopic capsule is also provided. The method includes a determination of a position and/or orientation of the endoscopic capsule. At least one sensor coil pair is arranged outside of the endoscopic capsule. The at least one sensor coil pair includes a first sensor coil and a second sensor coil. The first sensor coil and the second sensor coil are electrically connected to one another and arranged at locations with the same second magnetic flux density.

In one embodiment, the first sensor coil and the second sensor coil are connected to one another such that voltages induced by the first magnetic field are compensated.

In another embodiment, the first sensor coil and the second sensor coil may be connected to one another such that the voltages induced by the second magnetic field are compensated.

The first magnetic field may be generated by a driving coil, and the first sensor coil and the second sensor coil may be arranged symmetrically relative to a coil axis of the driving coil.

The second magnetic field may be generated by an energy coil, and the first sensor coil and the second sensor coil may be arranged symmetrically relative to the coil axis of the energy coil.

### BRIEF DESCRIPTION OF THE DRAWINGS

FIG. 1 shows a block diagram of an arrangement for navigating an endoscopic capsule according to the prior art;

FIG. 2 shows a top view of one embodiment of an arrangement having sensor coil pairs and an energy coil;

FIG. 3 shows a top view of one embodiment of an arrangement with sensor coil pairs, an energy coil and a driving coil; and

FIG. 4 shows an exemplary circuit diagram with a coil pair.

### DETAILED DESCRIPTION OF THE DRAWINGS

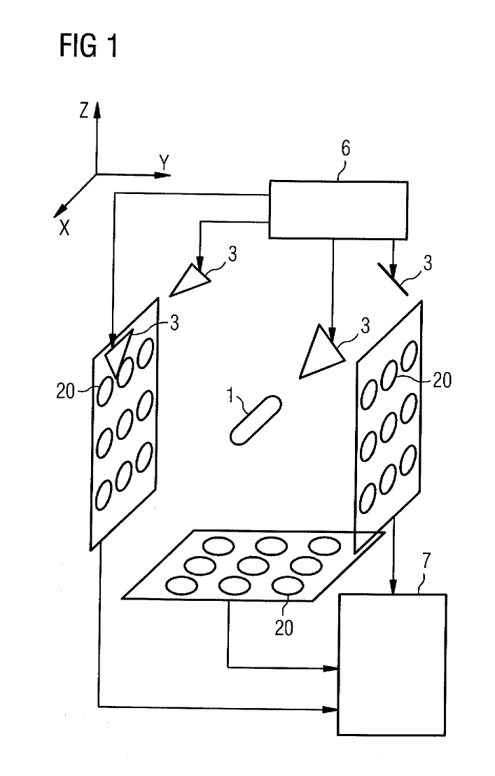

FIG. 1 shows a block diagram of an arrangement for navigating and determining a position of an endoscopic capsule 1, such as described, for example, in EP 1 967 137 A1.

The endoscopic capsule 1 is located within magnetic fields formed by driving coils 3. The position and orientation of the capsule 1 may be changed by changing the magnetic fields with the aid of a capsule driving unit 6. The magnetic fields of the driving coils 3 exert forces on a magnet inside of the capsule 1.

A current position and orientation of the capsule 1 may be determined from a magnetic alternating field formed in the capsule 1 using sensor coils 20 arranged in arrays in the x-, y- and z-direction. The sensor coils 20 are electrically connected to a position determination unit 7. The magnetic alternating field induces voltages in the sensor coils 20 that are evaluated for the position determination process. The magnetic alternating field is generated by a marker coil arranged in the capsule 1 (not shown). The electrical energy for establishing the alternating field may be provided by an energy coil.

Depending on the spatial position of the sensor coils 20, an interference voltage is also induced in the sensor coils 20. The interference voltage overlays the desired voltages for position determination and thus reduces the dynamic range of the position determination unit 7.

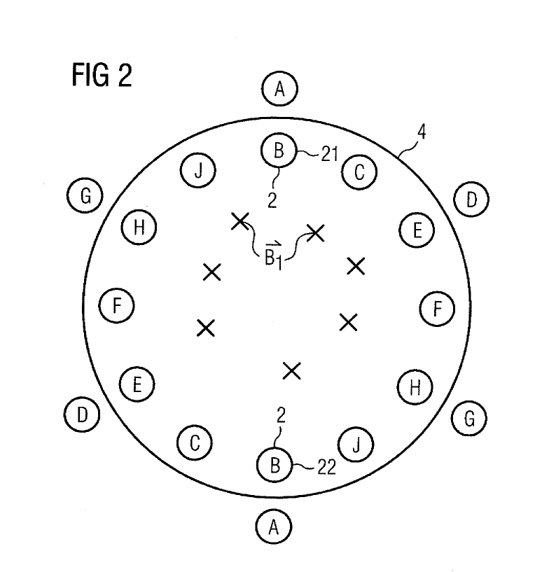

Sensor coil pairs 2 are formed according to FIG. 2. In each case, associated first and second sensor coils 21, 22 are designated by the same letters A, B, C, . . . J. The first and second sensor coils 21, 22 of the coil pair 2 are arranged in the magnetic field of an energy coil 4 such that at the site of the sensor coils 21, 22, a first magnetic flux density B̅₁ of the magnetic field of the energy coil 4 is equally great and similarly oriented. FIG. 2 shows the first magnetic flux density B̅₁ in the plane of projection. With the aid of the energy coil 4, a marker coil of the endoscopic capsule is supplied with electrical energy. The coils 21, 22 and 4 are shown in a top view, so that only a coil winding is visible.

An electrical connection of the first and second sensor coil 21, 22, according to FIG. 4, provides that voltages induced in the sensor coils 21, 22, which are to be attributed back to the magnetic field of the energy coil 4, are mutually cancelled out. To provide that the voltages induced by the endoscopic capsule in the two coils 21, 22 are as different as possible and not mutually cancelled out, the first and second coils 21, 22 are arranged as far removed from one another as possible. It is provided that the first magnetic flux density B̅₁ at the site of the first and second sensor coils 21, 22 is of equal magnitude. With the plurality of coil pairs 2 (A to J) according to FIG. 2, a symmetrical arrangement may be selected.

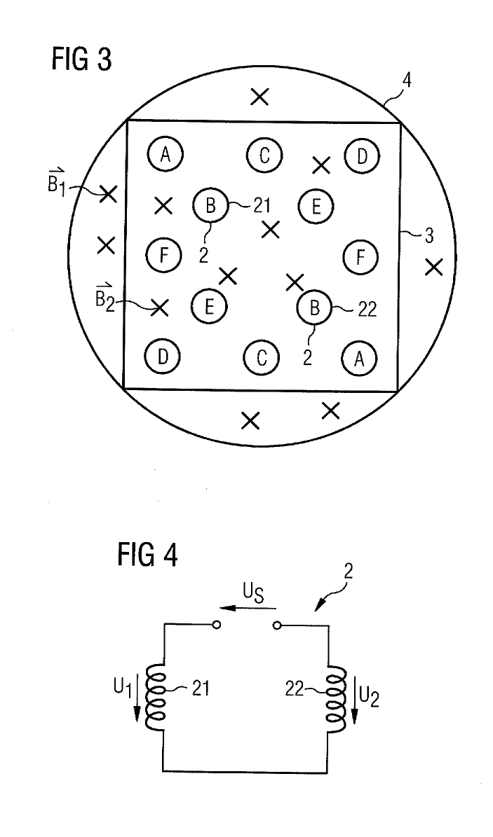

FIG. 3 shows an arrangement similar to FIG. 2. The only difference is that in FIG. 3, both a magnetic field of an energy coil 4 with a second magnetic flux B̅₁ and also a magnetic field of a driving coil 3 is present with a first magnetic flux density B̅₁ . The sensor coil pair 2 (A, B, . . . F) is passed through in this arrangement by the same first magnetic flux density B̅₁ and the same second magnetic flux density B̅₂, in each case. A centrally symmetrical arrangement further provides for a high dynamic range of the sensor voltages in the coil pairs 3 that are connected to one another.

FIG. 4 shows a circuit diagram of a coil pair 20. The first and second sensor coil 21, 22 are electrically connected in a counter-pole fashion to one another so that a first voltage U₁ and a second voltage U₂ induced by an interfering magnetic field are mutually compensated and even cancelled out with the same magnetic flux density at the site of the coils 21, 22. A sensor voltage U_{S} as the sum of the voltages U₁, U₂ induced by the interfering magnetic field is zero.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An arrangement for navigating an endoscopic capsule, the arrangement comprising:
an external first magnetic field with a first magnetic flux density for moving the endoscopic capsule; and
at least one sensor coil pair outside of the endoscopic capsule for determining a position, an orientation, or the position and the orientation of the endoscopic capsule, the at least one sensor coil pair comprising a first sensor coil and a second sensor coil,
wherein the first sensor coil and the second sensor coil are electrically connected to one another and arranged at locations with the same first magnetic flux density.

2. The arrangement as claimed in claim 1, further comprising an external second magnetic field with a second magnetic flux density for supplying power to a marker coil of the endoscopic capsule,
wherein the first sensor coil and the second sensor coil are arranged at locations with the same second magnetic flux density.

3. The arrangement as claimed in claim 2, wherein the first sensor coil and the second sensor coil are connected to one another such that voltages induced by the external first magnetic field are compensated.

4. The arrangement as claimed in claims 2, wherein the first sensor coil and the second sensor coil are connected to one another such that voltages induced by the external second magnetic field are compensated.

5. The arrangement as claimed in claim 1, further comprising a driving coil operable to generate the external first magnetic field,
wherein the first sensor coil and the second sensor coil are arranged symmetrically relative to a coil axis of the driving coil.

6. The arrangement as claimed in claim 2, further comprising an energy coil operable to generate the external second magnetic field,
wherein the first sensor coil and the second sensor coil are arranged symmetrically relative to a coil axis of the energy coil.

7. The arrangement as claimed in claims 3, wherein the first sensor coil and the second sensor coil are connected to one another such that voltages induced by the external second magnetic field are compensated.

8. The arrangement as claimed in claim 2, further comprising a driving coil operable to generate the external first magnetic field,
wherein the first sensor coil and the second sensor coil are arranged symmetrically relative to a coil axis of the driving coil.

9. The arrangement as claimed in claim 2, further comprising an energy coil operable to generate the external second magnetic field,
wherein the first sensor coil and the second sensor coil are arranged symmetrically relative to a coil axis of the energy coil.

10. An arrangement for navigating an endoscopic capsule, the arrangement comprising:
a source of an external magnetic field with a magnetic flux density for supplying power to a marker coil of the endoscopic capsule; and
at least one sensor coil pair outside of the endoscopic capsule for determining a position, an orientation, or the position and the orientation of the endoscopic capsule, the at least one sensor coil pair comprising a first sensor coil and a second sensor coil,
wherein the first sensor coil and the second sensor coil are electrically connected to one another and arranged at locations with the same magnetic flux density.

11. A method for navigating an endoscopic capsule through an external first magnetic field with a first magnetic flux density for moving the endoscopic capsule, the method comprising:
determining a position, an orientation, or the position and the orientation of the endoscopic capsule with at least one sensor coil pair outside of the endoscopic capsule, the at least one sensor coil pair including a first sensor coil and a second sensor coil,
wherein the first sensor coil and the second sensor coil are electrically connected to one another and arranged at locations with the same first magnetic flux density.

12. The method as claimed in claim 11, wherein the endoscopic capsule is navigated through an external second magnetic field with a second magnetic flux density for supplying power to a marker coil of the endoscopic capsule, and
wherein the first sensor coil and the second sensor coil are electrically connected to one another and arranged at locations with the same second magnetic flux density.

13. The method as claimed in claim 12, further comprising: compensating for voltages induced by the external first magnetic field by connection to one another of the first sensor coil and the second sensor coil.

14. The method as claimed in claim 12, further comprising: compensating for voltages induced by the external second magnetic field by connection to one another of the first sensor coil and the second sensor coil.

15. The method as claimed in claim 11, wherein the external first magnetic field is generated by a driving coil, and
wherein the first sensor coil and the second sensor coil are arranged symmetrically relative to a coil axis of the driving coil.

16. The method as claimed in claim 12, wherein the external second magnetic field is generated by an energy coil, and
wherein the first sensor coil and the second sensor coil are arranged symmetrically relative to a coil axis of the energy coil.

17. The method as claimed in claim 13, further comprising compensating for voltages induced by the external first magnetic field by connection to one another of the first sensor coil and the second sensor coil.

18. The method as claimed in claim 13, wherein the external first magnetic field is generated by a driving coil, and
wherein the first sensor coil and the second sensor coil are arranged symmetrically relative to a coil axis of the driving coil.

19. The method as claimed in claim 13, wherein the external second magnetic field is generated by an energy coil, and
wherein the first sensor coil and the second sensor coil are arranged symmetrically relative to a coil axis of the energy coil.

20. The method as claimed in claim 14, wherein the external second magnetic field is generated by an energy coil, and
wherein the first sensor coil and the second sensor coil are arranged symmetrically relative to a coil axis of the energy coil.
